(19) United States
(12) Patent Application Publication
(10) Pub. No.: US 2020/0164223 A1
(43) Pub. Date: May 28, 2020

(54) Laser Device for Treatment of Wounds

(71) Applicant: Vulcur Medtech ApS, l/anløse (DK)

(72) Inventor: Janus BEIERHOLM, Vanløse (DK)

(21) Appl. No.: 16/611,634

(22) PCT Filed: May 7, 2018
(86) PCT No.: PCT/DK2018/050097

§ 371 Date: Nov. 7, 2019

(30) Foreign Application Priority Data
May 8, 2017 (DK) PA 2017 00286

(51) Int. Cl. A61N 5/06; A61L 2/24; A61L 2/00

(52) U.S. Cl. CPC A61N 5/0624; A61N 5/0616; A61N 2005/067; A61L 2/0058; A61L 2/24

(57) ABSTRACT

The present invention relates to a device and a method for treatment or disinfection of a volume comprising bacteria in the vicinity of cells, such as a laser device and the use of the laser device for the treatment of wounds. In particular, it relates to the treatment of chronic wounds.

The present invention relates to a device and a method for treatment or disinfection of a volume comprising bacteria in the vicinity of cells, such as a laser device and the use of the laser device for the treatment of wounds. In particular, it relates to the treatment of chronic wounds, biofilms associated with chronic wounds, other chronic infections associated with biofilms and surgical site infections.

### TECHNICAL BACKGROUND

Regeneration and tissue repair after a tissue lesion happen in overlapping stages consisting of a hemostasis phase, an inflammatory phase, a proliferative phase and a remodeling phase.

In the hemostasis phase the wound is closed by clotting that stops bleeding. After hemostasis is established, blood vessels dilate and let antibodies, white blood cells, nutrients and other elements, that prevents infection and promote healing, into the wound. Tissue regeneration happens in the proliferative phase by cell proliferation and synthesis of the elements that make up extracellular matrix. In the last remodeling phase tensile strength is enhanced and scar thickness is reduced.

A wound becomes infected when microorganisms—especially bacteria—colonize the wound and delay healing or deteriorate the wound. Wound infections can arise when wounds are contaminated by bacteria or when the immune system isn't able to cope with normal bacterial growth. Surgical site infections are prevalent and can be life-threatening. Infections can also lead to chronic wounds (i.e. wounds not having healed within three months).

Research indicates that bacterial and fungal cells can exist in surface-attached clusters called biofilms. The bacteria attach to a solid surface, proliferate and form microcolonies that produce extracellular polymeric substances. Biofilms exist in most chronic wounds and induce resistance to antibiotics and biocides and increase intracellular survival rate. These properties of biofilm are hypothesized to be caused by poor antibiotic penetration, nutrient limitation, slow growth, adaptive stress responses, and formation of phenotypic variants.

The infection can spread to surrounding tissue if a clump of biofilm detaches from the original cluster and contaminates surrounding surfaces.

In normal wound healing the inflammatory phase lasts only a few days before progression to the proliferative phase. Bacterial biofilms can target many of the major inflammatory agents and cause a prolonged inflammatory phase of healing.

Biofilms are also present in numerous other types of chronic infections including airway infections in patients with cystic fibrosis and implant- and catheter-associated infections and are therefore associated with many deaths.

The techniques currently used to treat chronic wounds fail to target in depth bacterial biofilms effectively. In conventional chronic-wound treatment 65% of the patients are expected to be cured within the first 25 weeks.

The TIME model is commonly used to treat chronic wounds. It consists of debridement, wound cleansing, negative pressure wound therapy, treatment of infection and inflammation, balance of moisture in the wound and wound edge assessment.

There is a wide range of techniques available for debridement, which is removal of dead tissue and foreign matter. The most direct one is surgical excision, but mechanical (e.g. a saline-moistened gauze or saline irrigation), autolytic (e.g. an occlusive dressing), enzymatic and biological methods (such as maggots) can also be used. The removed tissue is most often the tissue with the highest bacterial count since wound healing is impaired by bacteria.

Wound cleansing removes cellular debris and surface pathogens and causes wound hydration. The preferred method is wound irrigation which is a steady flow of a solution across an open wound. Combined with debridement, irrigation is an important step in facilitating progression from the inflammatory to the proliferative phase of wound healing. This is done by clearing out debris that can impede healing.

Negative pressure wound therapy includes application of a wound dressing through which a negative pressure is applied. The technique is thought to remove wound exudate and infectious material, promote granulation tissue formation and perfusion and draw wound edges together. There is only limited evidence available at the moment to support the efficacy of negative pressure wound therapy.

Treatment of infections or inflammation unrelated to infection includes topical antimicrobials and systemic antibiotics. The choice of treatment depends on the type of microbial burden. Biofilms can be definitively detected by advanced microscopy or specialized culture techniques. The current strategy for treating biofilm involves debridement and cleansing to physically disrupt and remove the biofilm and topical antimicrobials to kill microorganisms and prevent further wound contamination.

Silver dressings have been used extensively as a topical antimicrobial dressing, but recent studies have concluded that there is insufficient evidence to show that silver dressings improve healing rates.

Balance of moisture includes assessment and management of wound fluid. Either excessive or insufficient exudate production may inhibit wound healing processes in chronic wounds.

Different dressing can provide appropriate moisture balance, prevent maceration of the skin edges and leakage. Protease-modulating dressings can control wound proteases that denature growth factors.

Epithelial edge advancement includes assessment and management of nonadvancing or undermining wound edges and the condition of the surrounding skin.

Edge advancement therapies inter alio include:
Low-level gas laser therapy (helium neon or gallium arsenide) which has been used to increase proliferation and migration of cells. There is limited evidence of benefit in using low-level gas laser therapy to treat chronic wounds.
Electromagnetic therapy which provides a continuous or pulsed electromagnetic field, that is thought to induce cellular proliferation. Recent research provides no strong evidence of an increase in healing rate when electromagnetic therapy is used.
Phototherapy which has been proposed as a therapy for wound healing. There is currently no evidence to support its benefit and safety.
Ultrasonic therapy that is hypothesized to stimulate cellular activity within the wound bed. There is no evidence of benefit associated with ultrasonic therapy on venous leg ulcers or pressure ulcers.
Autologous platelet-rich plasma gel which consists of cytokines, growth factors and a fibrin scaffold derived from the patient's own blood. A recent review showed that complete and partial wound healing was improved compared to control wound care, but the randomized controlled trials used were of low quality.

### SUMMARY OF THE INVENTION

There is a need for alternative treatment of wounds, in particular chronic wounds. In particular, there is a need for non-invasive treatment of wounds, which does not require any administration of any active substances.

This need is met by aspects and embodiments of the present invention as provided in the claims. Additional aspects and embodiments are provided below.

According to an aspect, the invention concerns a device for treatment or disinfection of a volume comprising bacteria in the vicinity of cells, said device comprising:
a. Means for generating a beam of electromagnetic radiation, said means preferably comprising a laser;
b. Optionally means for spreading said beam of electromagnetic radiation, said means for spreading preferably comprising a diverging lens;
c. Means for collimating said beam of electromagnetic radiation, said means for collimating preferably comprising a converging lens, thereby providing a beam of collimated electromagnetic radiation;
d. Means for focusing said beam of collimated electromagnetic radiation, said means for focusing preferably comprising at least one focusing lens, wherein said means for focusing allows focusing said beam of collimated electromagnetic radiation in at least one focal volume inside said volume to be treated or disinfected;

Wherein said device comprises means allowing changing the position of said at least one focal volume inside said volume to be treated or disinfected; and

Wherein said device is adapted to allow eradicating or harming said bacteria with said electromagnetic radiation while leaving said cells substantially unharmed, by allowing said electromagnetic radiation to provide sufficient energy in said at least one focal volume to eradicate or harm said bacteria while providing insufficient energy to substantially harm said cells.

According to another aspect, the invention concerns a device for treatment or disinfection of a volume comprising bacteria in the vicinity of cells, said device comprising:
e. Means for generating collimated electromagnetic radiation;
f. Means for focusing said collimated electromagnetic radiation, said means for focusing preferably comprising at least one focusing optical lens, wherein said means for focusing allows focusing said electromagnetic radiation in at least one focal volume inside said volume to be treated or disinfected;

Wherein said device comprises means allowing changing the position of said at least one focal volume inside said volume to be treated or disinfected; and

Wherein said device is adapted to allow eradicating or harming said bacteria with said electromagnetic radiation while leaving said cells substantially unharmed, by allowing said electromagnetic to provide sufficient energy in said at least one focal volume to eradicate or harm said bacteria while providing insufficient energy to substantially harm said cells.

According to an aspect, the invention concerns a use of a device according to the invention for treatment or prophylaxis.

A use of a device according to any of the preceding device claims for in-vitro or non-medical purposes, such as cosmetic purposes.

According to an aspect, the invention concerns a method for the treatment or disinfection of a volume comprising bacteria in the vicinity of cells, said method comprising transmitting electromagnetic radiation to at least one focal volume inside said volume to be treated or disinfected by allowing said electromagnetic to provide sufficient energy in said at least one focal volume to eradicate or harm said bacteria while providing insufficient energy to substantially harm said cells.

### DETAILED DISCLOSURE

Embodiments of the present invention are provided in the claims. Additional embodiments are provided below and in the figures.

According to an embodiment, the invention concerns a device for treatment or disinfection of a volume comprising bacteria in the vicinity of cells, said device comprising:
a. Means for generating a beam of electromagnetic radiation, said means preferably comprising a laser;
b. Optionally means for spreading said beam of electromagnetic radiation, said means for spreading preferably comprising a diverging lens;
c. Means for collimating said beam of electromagnetic radiation, said means for collimating preferably comprising a converging lens, thereby providing a beam of collimated electromagnetic radiation;
d. Means for focusing said beam of collimated electromagnetic radiation, said means for focusing preferably comprising at least one focusing lens, wherein said means for focusing allows focusing said beam of collimated electromagnetic radiation in at least one focal volume inside said volume to be treated or disinfected;

Wherein said device comprises means allowing changing the position of said at least one focal volume inside said volume to be treated or disinfected; and

Wherein said device is adapted to allow eradicating or harming said bacteria with said electromagnetic radiation while leaving said cells substantially unharmed, by allowing said electromagnetic radiation to provide sufficient energy in said at least one focal volume to eradicate or harm said bacteria while providing insufficient energy to substantially harm said cells.

According to a preferred embodiment, optical lenses are used as the lenses.

According to an embodiment, the means b. are situated between the means a. and c.

According to an embodiment, the means c. are situated between the means b. and d.

According to an embodiment, the means c. are situated between the means a. and d.

According to an embodiment, the invention concerns the device, wherein said means b. comprises a diverging or negative lens.

According to an embodiment, the invention concerns the device, wherein said means c. comprises a condenser lens or converging or positive lens or a collimator.

According to an embodiment, the invention concerns the device, wherein said means d. comprises a plurality of lenses, such as a micro array lens.

According to an embodiment, the invention concerns the device, wherein at least one of said means b., c., and d. allows changing the position of said focal volume inside said volume to be treated or disinfected.

According to an embodiment, the invention concerns the device, wherein said device comprises means for changing the distance between the means c. and the means d., thereby allowing changing the position of said focal volume inside said volume to be treated or disinfected. This allows affecting bacteria in different depths of the volume to be treated or disinfected.

According to an embodiment, the invention concerns the device, wherein said device comprises means for changing the position of said means d. with respect to said collimated electromagnetic direction in at least two independent directions, thereby allowing changing the position of said focal volume inside said volume to be treated or disinfected. Preferably these two independent directions are substantially perpendicular to each other. In a preferred embodiment, the two independent directions are substantially perpendicular to the direction of propagation of the collimated electromagnetic radiation.

According to an embodiment, the invention concerns a device for treatment or disinfection of a volume comprising bacteria in the vicinity of cells, said device comprising:
e. Means for generating collimated electromagnetic radiation;
f. Means for focusing said collimated electromagnetic radiation, said means for focusing preferably comprising at least one focusing optical lens, wherein said means for focusing allows focusing said electromagnetic radiation in at least one focal volume inside said volume to be treated or disinfected;

Wherein said device comprises means allowing changing the position of said at least one focal volume inside said volume to be treated or disinfected; and

Wherein said device is adapted to allow eradicating or harming said bacteria with said electromagnetic radiation while leaving said cells substantially unharmed, by allowing said electromagnetic to provide sufficient energy in said at least one focal volume to eradicate or harm said bacteria while providing insufficient energy to substantially harm said cells.

According to an embodiment, the invention concerns the device, wherein said focal volume has a volume of 1-10.000 μm³, preferably 2-5000 μm³, more preferred 3-3000 μm³, preferably 5-2000 μm³, more preferred 10-1000 μm³, preferably 20-500 μm³, more preferred 30-400 μm³, preferably 50-200 μm³, more preferred about 100 μm³.

The focal volume is the area of the focal spot times the focus depth. The focus depth may be calculated as two times the Rayleigh length, Z_{R}. The Rayleigh length, Z_{R}, may be calculated as Z_{R}=π(ω₀)²/λ. The beam waist, ω₀, is the radius of the area of the focal spot. The wavelength of the electromagnetic radiation is λ. The area of the focal spot may be calculated as π(ω₀)² for a circular focal spot, and as π(length of the minor axis)(length of major axis)/4 for an elliptical focal spot. In cases where the beam does not have a circular symmetry, it may be relevant to consider another measure than the beam waist, e.g. the full width at half maximum, the D86 width, the 1/e² width or the D4σ width.

According to an embodiment, the invention concerns the device, wherein said focal volume has a volume of at least 1 μm³, preferably at least 2 μm³, more preferred at least 3 μm³, preferably at least 5 μm³, more preferred at least 10 μm³, preferably at least 20 μm³, more preferred at least 50 μm³, preferably at least 100 μm³, more preferred at least 200 μm³, preferably at least 300 μm³, more preferred at least 500 μm³, preferably at least 1000 μm³, more preferred at least 2000 μm³.

According to an embodiment, the invention concerns the device, wherein said focal volume has a volume of less than 5000 μm³, preferably less than 3000 μm³, more preferred less than 2000 μm³, preferably less than 1000 μm³, more preferred less than 500 μm³, preferably less than 300 μm³, more preferred less than 200 μm³, preferably less than 100 μm³, more preferred less than 50 μm³, preferably less than 30 μm³, more preferred less than 20 μm³, preferably less than 10 μm³, more preferred less than 5 μm³.

According to an embodiment, the invention concerns the device, wherein the focus depth is 0.5-500 μm, preferably 2-200 μm, more preferred 3-100 μm, preferably 5-50 μm, more preferred 10-20 μm.

According to an embodiment, the invention concerns the device, wherein the focus spot is an area of 0.05-100 μm², preferably 0.1-50 μm², more preferred 0.2-20 μm², preferably 0.5-10 μm², more preferred 1-5 μm².

According to an embodiment, the invention concerns the device, wherein said volume to be treated or disinfected comprises at least part of a wound, such as a chronic wound.

According to an embodiment, the invention concerns the device, wherein said device has access to 3D information about the distribution of said wound in the tissue thereby allowing focusing the electromagnetic radiation inside said wound.

According to an embodiment, the invention concerns the device, wherein said device further comprises means for moving said volume to be treated or disinfected with respect to said focal volume thereby changing the position of said focal volume with respect to said volume to be treated or disinfected.

According to an embodiment, the invention concerns the device, wherein said device further comprises means for keeping said volume to be treated or disinfected in a fixed position with respect to said device.

According to an embodiment, the invention concerns the device, wherein said device allows changing the position of said focal volume inside said volume to be treated or disinfected in a helical and/or zigzag pattern.

According to an embodiment, the invention concerns the device, wherein said device allows changing the position of said focal volume, allowing said focal volume to travel in lines through said volume to be treated or disinfected with a determined spacing between said lines.

According to an embodiment, the invention concerns the device, wherein said determined spacing is 1-200 μm, preferably 2-100 μm, more preferred 3-50 μm, preferably 5-40 μm, more preferred 10-30 μm, preferably 15-25 μm, more preferred about 20 μm.

According to an embodiment, the invention concerns the device, wherein said electromagnetic radiation has a wavelength of 100-3000 nm, preferably 200-2500 nm, more preferred 300-2000 nm, preferably 500-1500 nm, more preferred 700-1400 nm, preferably 800-1300 nm, more preferred 900-1200 nm, preferably 1000-1125 nm, more preferred 1025-1100 nm, preferably 1050-1080 nm, more preferred 1060-1070 nm, preferably about 1064 nm.

According to an embodiment, the invention concerns the device, wherein said device allows said electromagnetic radiation to be provided as electromagnetic pulses.

According to an embodiment, the invention concerns the device, wherein said electromagnetic pulses have duration of 0.01-1000 ns, more preferred 0.05-100 ns, preferably 0.1-20 ns, more preferred 0.5-10 ns, preferably 1-8 ns, more preferred 2-6 ns, preferably 3-5 ns, more preferred about 4 ns.

According to an embodiment, the invention concerns the device, wherein the electromagnetic pulses have duration sufficient to eradicate or harm bacteria while having duration insufficient to substantially harm cells.

According to an embodiment, the invention concerns the device, wherein each of said electromagnetic pulses provides an amount of energy of 1-10.000 nJ, preferably 5-5.000 nJ, more preferred 10-2500 nJ, preferably 20-1000 nJ, more preferred 30-500 nJ, preferably 40-100 nJ, more preferred about 50 nJ in each of said at least one focal volume.

According to an embodiment, the invention concerns the device, wherein each of said electromagnetic pulses provides an amount of energy of less than 10.000 nJ, preferably less than 5.000 nJ, more preferred less than 2500 nJ, preferably less than 1000 nJ, more preferred less than 500 nJ, preferably less than 100 nJ, more preferred about less than 50 nJ in each of said at least one focal volume.

According to an embodiment, the invention concerns the device, wherein said device allows providing said electromagnetic pulses with a frequency of 1-100 kHz, more preferred 5-50 kHz, preferably 10-40 kHz, more preferred 15-30 kHz, preferably about 20 kHz. With a frequency of 20 kHz, 20.000 pulses may be provided every second.

According to an embodiment, the invention concerns the device, wherein the electromagnetic pulses are focused inside said volume to be treated or disinfected with a distance of 1-200 μm, preferably 2-100 μm, more preferred 3-50 μm, preferably 5-40 μm, more preferred 10-30 μm, preferably 15-25 μm, more preferred about 20 μm between said pulses.

According to an embodiment, the invention concerns the device, wherein said volume to be treated or disinfected has a surface, and wherein said at least one focal volume is at least a distance of 1 μm, more preferred at least 2 μm, preferably at least 5 μm, more preferred at least 10 μm, preferably at least 20 μm, more preferred at least 50 μm, preferably at least 100 μm, more preferred at least 200 μm, from said surface.

According to an embodiment, the invention concerns the device, wherein said volume to be treated or disinfected has a surface, and wherein said at least one focal volume is a distance of 1-500 μm, more preferred 5-300 μm, preferably 10-200 μm, more preferred 40-100 μm, from said surface.

According to an embodiment, the invention concerns the device, wherein the focal length is 1-100 mm, more preferred 2-50 mm, preferably 3-30 mm, more preferred 4-20, preferably 5-10 mm. The focal length may be defined as the distance between the center of the means for focusing said collimated electromagnetic radiation and the center of the focal volume.

According to an embodiment, the invention concerns a use of a device according to the invention for treatment or prophylaxis.

According to an embodiment, the invention concerns the use, wherein the subject is human or animal, preferably a mammal.

According to an embodiment, the invention concerns the use, wherein said volume to be treated or disinfected is part of the body, such as a limb, a leg or an arm.

According to an embodiment, the invention concerns the use of the device for topical use.

According to an embodiment, the invention concerns the use of the device for non-invasive use.

According to an embodiment, the invention concerns the use of the device without using medicaments.

According to an embodiment, the invention concerns a use of a device according to the invention for in-vitro or non-medical purposes, such as cosmetic purposes.

According to an embodiment, the invention concerns a method for the treatment or disinfection of a volume comprising bacteria in the vicinity of cells, said method comprising transmitting electromagnetic radiation to at least one focal volume inside said volume to be treated or disinfected by allowing said electromagnetic to provide sufficient energy in said at least one focal volume to eradicate or harm said bacteria while providing insufficient energy to substantially harm said cells.

According to an embodiment, the invention concerns the method, wherein said volume to be treated or disinfected has a surface, and wherein said at least one focal volume is at least a distance of 1 μm, more preferred at least 2 μm, preferably at least 5 μm, more preferred at least 10 μm, preferably at least 20 μm, more preferred at least 50 μm, preferably at least 100 μm, more preferred at least 200 μm, from said surface.

According to an embodiment, the invention concerns the method, wherein said volume to be treated or disinfected has a surface, and wherein said at least one focal volume is a distance of 1-500 μm, more preferred 5-300 μm, preferably 10-200 μm, more preferred 40-100 μm, from said surface.

According to an embodiment, the invention concerns the method, wherein said electromagnetic radiation is generated by a laser.

According to an embodiment, the invention concerns the method, wherein said laser is operated in a continuous or pulsed mode.

According to an embodiment, the invention concerns the method, wherein said at least one focal volume is moved within said volume to be treated or disinfected with a velocity allowing said electromagnetic to provide sufficient energy in said at least one focal volume to eradicate or harm said bacteria while providing insufficient energy to substantially harm said cells.

According to an embodiment, the invention concerns the method, wherein said volume to be treated or disinfected comprises at least part of a wound, such as a chronic wound.

According to an embodiment, the invention concerns the method, wherein said the electromagnetic radiation is focused inside said wound.

According to an embodiment, the invention concerns the method, wherein said method comprises changing the position of said focal volume inside said volume to be treated or disinfected in a helical and/or zigzag pattern.

According to an embodiment, the invention concerns the method, wherein said method comprises changing the position of said focal volume, allowing said focal volume to travel in lines through said volume to be treated or disinfected with a determined spacing between said lines.

According to an embodiment, the invention concerns the method, wherein said determined spacing is 1-200 μm, preferably 2-100 μm, more preferred 3-50 μm, preferably 5-40 μm, more preferred 10-30 μm, preferably 15-25 μm, more preferred about 20 μm.

According to an embodiment, the invention concerns the method, wherein said electromagnetic radiation has a wavelength of 100-3000 nm, preferably 200-2500 nm, more preferred 300-2000 nm, preferably 500-1500 nm, more preferred 700-1400 nm, preferably 800-1300 nm, more preferred 900-1200 nm, preferably 1000-1125 nm, more preferred 1025-1100 nm, preferably 1050-1080 nm, more preferred 1060-1070 nm, preferably about 1064 nm.

According to an embodiment, the invention concerns the method, wherein said electromagnetic radiation is provided as electromagnetic pulses.

According to an embodiment, the invention concerns the method, wherein said electromagnetic pulses have duration of 0.1-20 ns, more preferred 0.5-10 ns, preferably 1-8 ns, more preferred 2-6 ns, preferably 3-5 ns, more preferred about 4 ns.

According to an embodiment, the invention concerns the method, wherein the electromagnetic pulses have duration sufficient to eradicate or harm bacteria while having duration insufficient to substantially harm cells.

According to an embodiment, the invention concerns the method, wherein each of said electromagnetic pulses provides an amount of energy of 1-10.000 nJ, preferably 5-5.000 nJ, more preferred 10-2500 nJ, preferably 20-1000 nJ, more preferred 30-500 nJ, preferably 40-100 nJ, more preferred about 50 nJ in each of said at least one focal volume.

According to an embodiment, the invention concerns the method, wherein each of said electromagnetic pulses provides an amount of energy of less than 10.000 nJ, preferably less than 5.000 nJ, more preferred less than 2500 nJ, preferably less than 1000 nJ, more preferred less than 500 nJ, preferably less than 100 nJ, more preferred about less than 50 nJ in each of said at least one focal volume.

According to an embodiment, the invention concerns the method, wherein said electromagnetic pulses are provided with a frequency of 1-100 kHz, more preferred 5-50 kHz, preferably 10-40 kHz, more preferred 15-30 kHz, preferably about 20 kHz. With a frequency of 20 kHz, 20.000 pulses are provided every second.

According to an embodiment, the invention concerns the method, wherein the electromagnetic pulses are focused inside said volume to be treated or disinfected with a distance of 1-200 μm, preferably 2-100 μm, more preferred 3-50 μm, preferably 5-40 μm, more preferred 10-30 μm, preferably 15-25 μm, more preferred about 20 μm between said pulses.

According to an embodiment, the invention concerns the method, wherein said focal volume is moved around in said volume to be treated or disinfected, thereby providing treatment and/or disinfection of all or substantially all of said volume to be treated or disinfected.

According to an embodiment, the invention concerns the method, wherein said focal volume is moved around multiple times in said volume to be treated or disinfected in multiple passes. This embodiment may Increase the probability that the volume is rendered disinfected. According to an embodiment, this may be done by covering the same pattern of movement of said at least one focal volume, with a small distance between said patterns for each repeated pass.

According to an embodiment, the invention concerns the method, wherein the content of said volume comprising bacteria is substantially solid or non-liquid or non-fluid. The method is particularly applicable for tissue wherein the volume subjected to treatment has a high viscosity or is not a fluid; thereby ensuring bacteria in the volume have a low degree of mobility during the treatment.

### FIGURES

FIG. 1 shows a schematic view of a device for treatment or disinfection of a volume comprising bacteria in the vicinity of cells according to an embodiment of the invention. The device comprises a diverging lens (104) for spreading the electromagnetic radiation (102) provided by a laser (not shown), and a converging lens acting as a collimator (106) for the electromagnetic radiation, and thereby providing a beam of collimated electromagnetic radiation (107). A micro array lens (108) focuses the collimated electromagnetic radiation in a focal volume (110) inside the volume subjected to treatment or being disinfected, where the focal length (112) is the distance between the micro array lens and the focus point (111) at the center of the focal volume (110). The device allows changing the distance between the converging lens (106) and the micro array lens (108), i.e. along the axis indicated with z, as well as the position of the micro array lens in the x-y plane, thereby changing the position of the focal volume (110) inside the volume comprising bacteria.

FIG. 2 is a schematic representation of the movement of the focal volume inside the xyz volume. The device allows changing the position of the focal volume in a helical pattern (214), where the preferred distance between the lines in the z direction is 20 μm (216). The electromagnetic radiation may operated in a continuous (218) or pulsed mode (220), wherein the pulses have duration sufficient to eradicate or harm the bacteria while having duration insufficient to substantially harm the cells.

FIG. 3 is a schematic view along the z-axis within the area of a micro array lens (308) according to an embodiment of the invention. Collimated electromagnetic radiation (not shown) is passed through the lens (308). The focal volume (310) is the area of the focal spot times the focus depth (322), where the area of the focal spot is calculated as π(ω₀)² if circular, and the focus depth is calculated as two times the Rayleigh length, Z_{R}. The Rayleigh length, Z_{R}, is calculated as Z_{R}=π(ω₀)²/λ where the beam waist (324), ω₀, is the radius of the area of the focal spot, and λ is the wavelength of the electromagnetic radiation.

FIG. 4 is a photograph of the device for treatment or disinfection of a volume comprising bacteria in the vicinity of cells according to an embodiment of the invention. The photograph shows a leg fixed to the device, but any infected part of the body of humans or animals is contemplated to be fixed with respect to the device.

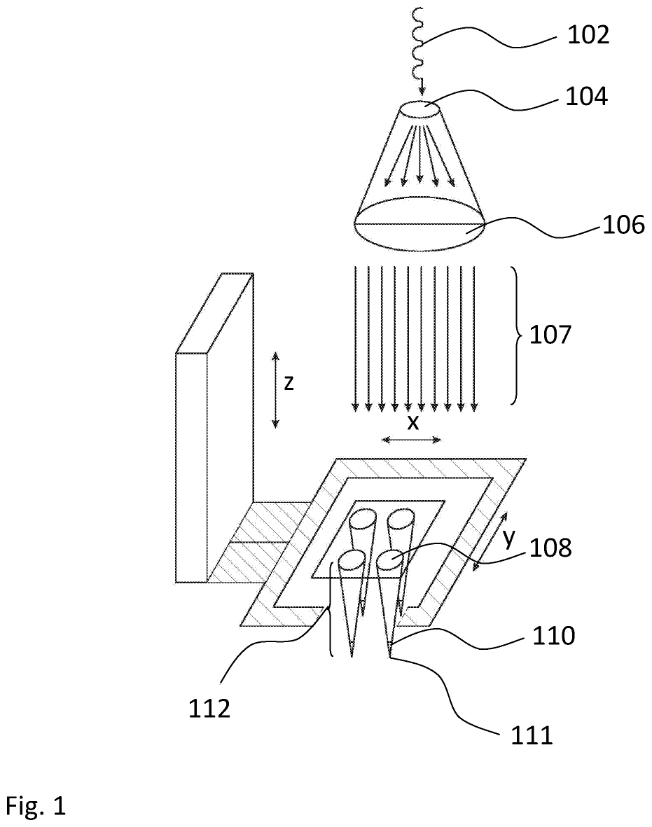

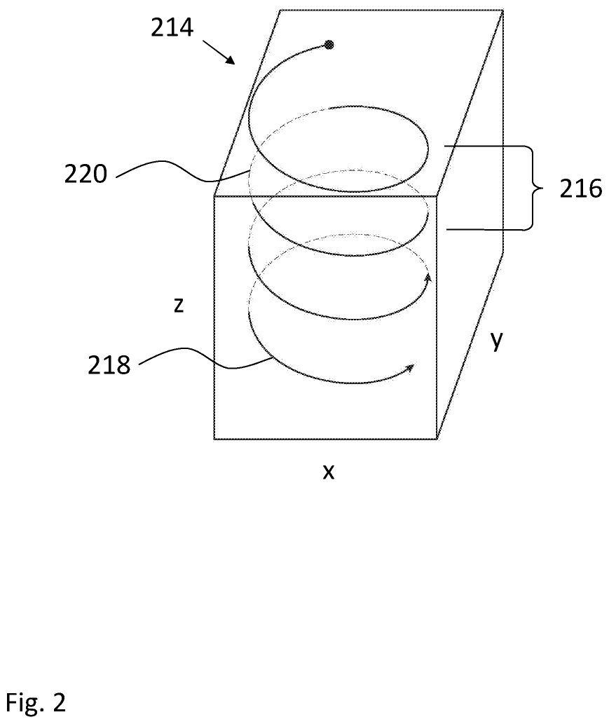

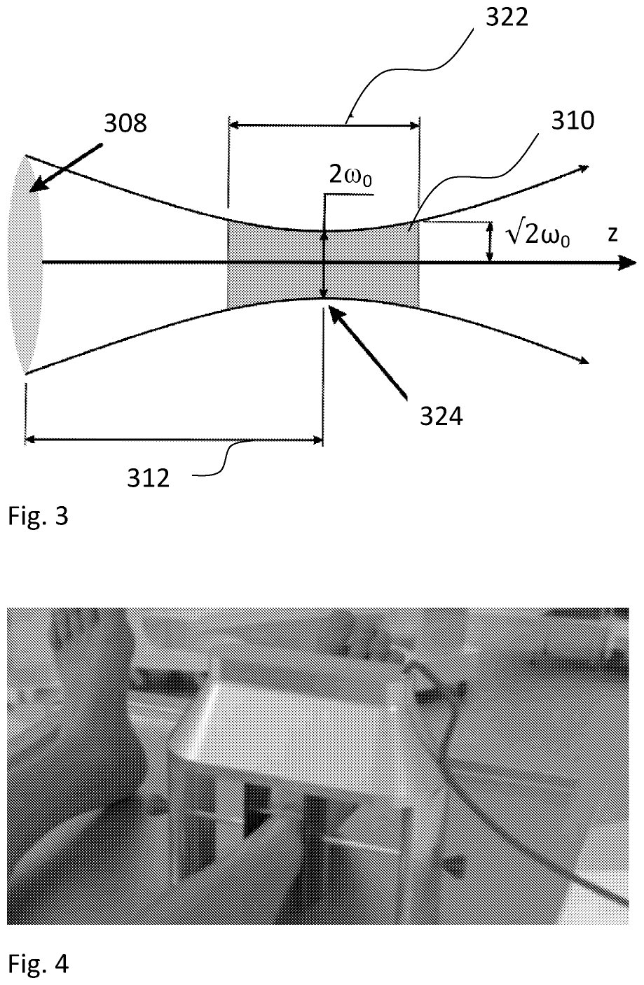

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure).

### EXAMPLES

### Example 1

Purpose: To identify the relative amount of energy required for killing a vital E. coli (Escherichia coli) cell and a vital human fibroblast cell, respectively.

Method: Human fibroblast cells and E. coli cells were stained with SYTO 9 dye and propidium iodide. A sample was prepared by mixing stained human fibroblast cells, stained E. coli cells and low-melt agarose, providing embedment of cells in the low-melt agarose. A drop was taken from the sample and placed on a glass slide. The glass slide was placed under a conventional confocal microscope equipped with a 405-nm laser. Using a standard confocal setup, a UV ablation method was utilized to selectively induce cellular death and to visualize single-cell responses in a dose-dependent manner. Vital cells were identified as being green, whereas non-vital (dead) cells were identified as being red. A vital E. coli cell was identified and the 405-nm laser was engaged. The 405-nm laser intensity was initially set low and the laser intensity was increased until one E. coli cell was killed using one laser pulse. Afterwards, starting from the laser intensity sufficient to kill one E. coli cell, the intensity was decreased until one E. coli cell was no longer killed using one laser pulse. For each new laser intensity a new E. coli cell was identified and used. The procedure was repeated for human fibroblast cells. The applied laser power output is instrument-specific and will differ for every confocal setup, but may be adjusted as described here to achieve the desired effect. Subsequently the identified effect or power setting may be used to eradicate or harm bacteria with the electromagnetic radiation while leaving cells substantially unharmed.

Results: Using the 405-nm laser, E. coli cells were generally killed when applying laser intensities of 35% of full power with one single laser pulse, whereas human fibroblast cells in general required laser intensities of 100% of full power at least 50 times in order to kill the cells.

Conclusion: The amount of energy required for killing an E. coli cell is considerably less that the amount of energy required for killing a human fibroblast cell, likely in the order of about 1000 times less.

1. A device for treatment or disinfection of a volume comprising bacteria in the vicinity of cells, said device comprising:
a. Means for generating a beam of electromagnetic radiation, said means preferably comprising a laser;
b. Optionally means for spreading said beam of electromagnetic radiation, said means for spreading preferably comprising a diverging lens;
c. Means for collimating said beam of electromagnetic radiation, said means for collimating preferably comprising a converging lens, thereby providing a beam of collimated electromagnetic radiation;
d. Means for focusing said beam of collimated electromagnetic radiation, said means for focusing preferably comprising at least one focusing lens, wherein said means for focusing allows focusing said beam of collimated electromagnetic radiation in at least one focal volume inside said volume to be treated or disinfected;
Wherein said device comprises means allowing changing the position of said at least one focal volume inside said volume to be treated or disinfected; and
Wherein said device is adapted to allow eradicating or harming said bacteria with said electromagnetic radiation while leaving said cells substantially unharmed, by allowing said electromagnetic radiation to provide sufficient energy in said at least one focal volume to eradicate or harm said bacteria while providing insufficient energy to substantially harm said cells.

2. The device according to claim 1, wherein said means b. comprises a diverging or negative lens.

3. The device according to any of the preceding claims, wherein said means c. comprises a condenser lens or converging or positive lens or a collimator.

4. The device according to any of the preceding claims, wherein said means d. comprises a plurality of lenses, such as a micro array lens.

5. The device according to any of the preceding claims, wherein at least one of said means b., c., and d. allows changing the position of said focal volume inside said volume to be treated or disinfected.

6. The device according to any of the preceding claims, wherein said device comprises means for changing the distance between the means c. and the means d., thereby allowing changing the position of said focal volume inside said volume to be treated or disinfected.

7. The device according to any of the preceding claims, wherein said device comprises means for changing the position of said means d. with respect to said collimated electromagnetic direction in at least two independent directions, thereby allowing changing the position of said focal volume inside said volume to be treated or disinfected.

8. A device for treatment or disinfection of a volume comprising bacteria in the vicinity of cells, said device comprising:
e. Means for generating collimated electromagnetic radiation;
f. Means for focusing said collimated electromagnetic radiation, said means for focusing preferably comprising at least one focusing optical lens, wherein said means for focusing allows focusing said electromagnetic radiation in at least one focal volume inside said volume to be treated or disinfected;
Wherein said device comprises means allowing changing the position of said at least one focal volume inside said volume to be treated or disinfected; and
Wherein said device is adapted to allow eradicating or harming said bacteria with said electromagnetic radiation while leaving said cells substantially unharmed, by allowing said electromagnetic to provide sufficient energy in said at least one focal volume to eradicate or harm said bacteria while providing insufficient energy to substantially harm said cells.

9. The device according to any of the preceding claims, wherein said focal volume has a volume of 1-10.000 μm³, preferably 2-5000 μm³, more preferred 3-3000 μm³, preferably 5-2000 μm³, more preferred 10-1000 μm³, preferably 20-500 μm³, more preferred 30-400 μm³, preferably 50-200 μm³, more preferred about 100 μm³.

10. The device according to any of the preceding claims, wherein said focal volume has a volume of at least 1 μm³, preferably at least 2 μm³, more preferred at least 3 μm³, preferably at least 5 μm³, more preferred at least 10 μm³, preferably at least 20 μm³, more preferred at least 50 μm³, preferably at least 100 μm³, more preferred at least 200 μm³, preferably at least 300 μm³, more preferred at least 500 μm³, preferably at least 1000 μm³, more preferred at least 2000 μm³.

11. The device according to any of the preceding claims, wherein said focal volume has a volume of less than 5000 μm³, preferably less than 3000 μm³, more preferred less than 2000 μm³, preferably less than 1000 μm³, more preferred less than 500 μm³, preferably less than 300 μm³, more preferred less than 200 μm³, preferably less than 100 μm³, more preferred less than 50 μm³, preferably less than 30 μm³, more preferred less than 20 μm³, preferably less than 10 μm³, more preferred less than 5 μm³.

12. The device according to any of the preceding claims, wherein the focus depth is 0.5-500 μm, preferably 2-200 μm, more preferred 3-100 μm, preferably 5-50 μm, more preferred 10-20 μm.

13. The device according to any of the preceding claims, wherein the focus spot is an area of 0.05-100 μm², preferably 0.1-50 μm², more preferred 0.2-20 μm², preferably 0.5-10 μm², more preferred 1-5 μm².

14. The device according to any of the preceding claims, wherein said volume to be treated or disinfected comprises at least part of a wound, such as a chronic wound.

15. The device according claim 14, wherein said device has access to 3D information about the distribution of said wound in the tissue thereby allowing focusing the electromagnetic radiation inside said wound.

16. The device according to any of the preceding claims, wherein said device further comprises means for moving said volume to be treated or disinfected with respect to said focal volume thereby changing the position of said focal volume with respect to said volume to be treated or disinfected.

17. The device according to any of the preceding claims, wherein said device further comprises means for keeping said volume to be treated or disinfected in a fixed position with respect to said device.

18. The device according to any of the preceding claims, wherein said device allows changing the position of said focal volume inside said volume to be treated or disinfected in a helical and/or zigzag pattern.

19. The device according to any of the preceding claims, wherein said device allows changing the position of said focal volume, allowing said focal volume to travel in lines through said volume to be treated or disinfected with a determined spacing between said lines.

20. The device according to claim 19, wherein said determined spacing is 1-200 μm, preferably 2-100 μm, more preferred 3-50 μm, preferably 5-40 μm, more preferred 10-30 μm, preferably 15-25 μm, more preferred about 20 μm.

21. The device according to any of the preceding claims, wherein said electromagnetic radiation has a wavelength of 100-3000 nm, preferably 200-2500 nm, more preferred 300-2000 nm, preferably 500-1500 nm, more preferred 700-1400 nm, preferably 800-1300 nm, more preferred 900-1200 nm, preferably 1000-1125 nm, more preferred 1025-1100 nm, preferably 1050-1080 nm, more preferred 1060-1070 nm, preferably about 1064 nm.

22. The device according to any of the preceding claims, wherein said device allows said electromagnetic radiation to be provided as electromagnetic pulses.

23. The device according to claim 22, wherein said electromagnetic pulses have duration of 0.01-1000 ns, more preferred 0.05-100 ns, preferably 0.1-20 ns, more preferred 0.5-10 ns, preferably 1-8 ns, more preferred 2-6 ns, preferably 3-5 ns, more preferred about 4 ns.

24. The device according to claim 22 or 23, wherein the electromagnetic pulses have duration sufficient to eradicate or harm bacteria while having duration insufficient to substantially harm cells.

25. The device according to any of the claims 22-24, wherein each of said electromagnetic pulses provides an amount of energy of 1-10.000 nJ, preferably 5-5.000 nJ, more preferred 10-2500 nJ, preferably 20-1000 nJ, more preferred 30-500 nJ, preferably 40-100 nJ, more preferred about 50 nJ in each of said at least one focal volume.

26. The device according to any of the claims 22-25, wherein each of said electromagnetic pulses provides an amount of energy of less than 10.000 nJ, preferably less than 5.000 nJ, more preferred less than 2500 nJ, preferably less than 1000 nJ, more preferred less than 500 nJ, preferably less than 100 nJ, more preferred about less than 50 nJ in each of said at least one focal volume.

27. The device according to any of the claims 22-26, wherein said device allows providing said electromagnetic pulses with a frequency of 1-100 kHz, more preferred 5-50 kHz, preferably 10-40 kHz, more preferred 15-30 kHz, preferably about 20 kHz.

28. The device according to any of the claims 22-25, wherein the electromagnetic pulses are focused inside said volume to be treated or disinfected with a distance of 1-200 μm, preferably 2-100 μm, more preferred 3-50 μm, preferably 5-40 μm, more preferred 10-30 μm, preferably 15-25 μm, more preferred about 20 μm between said pulses.

29. The device according to any of the preceding claims, wherein said volume to be treated or disinfected has a surface, and wherein said at least one focal volume is at least a distance of 1 μm, more preferred at least 2 μm, preferably at least 5 μm, more preferred at least 10 μm, preferably at least 20 μm, more preferred at least 50 μm, preferably at least 100 μm, more preferred at least 200 μm, from said surface.

30. The device according to any of the preceding claims, wherein said volume to be treated or disinfected has a surface, and wherein said at least one focal volume is a distance of 1-500 μm, more preferred 5-300 μm, preferably 10-200 μm, more preferred 40-100 μm, from said surface.

31. The device according to any of the preceding claims, wherein the focal length is 1-100 mm, more preferred 2-50 mm, preferably 3-30 mm, more preferred 4-20, preferably 5-10 mm.

32. A use of a device according to any of the preceding claims for treatment or prophylaxis.

33. The use according to claim 32, wherein the subject is human or animal, preferably a mammal.

34. The use according to any of the claims 32-33, wherein said volume to be treated or disinfected is part of the body, such as a limb, a leg or an arm.

35. The use according to any of the claims 32-34, for topical use.

36. The use according to any of the claims 32-35, for non-invasive use.

37. The use according to any of the claims 32-36, for use without medicaments.

38. A use of a device according to any of the preceding device claims for in-vitro or non-medical purposes, such as cosmetic purposes.

39. A method for the treatment or disinfection of a volume comprising bacteria in the vicinity of cells, said method comprising transmitting electromagnetic radiation to at least one focal volume inside said volume to be treated or disinfected by allowing said electromagnetic to provide sufficient energy in said at least one focal volume to eradicate or harm said bacteria while providing insufficient energy to substantially harm said cells.

40. The method according to claim 39, wherein said volume to be treated or disinfected has a surface, and wherein said at least one focal volume is at least a distance of 1 μm, more preferred at least 2 μm, preferably at least 5 μm, more preferred at least 10 μm, preferably at least 20 μm, more preferred at least 50 μm, preferably at least 100 μm, more preferred at least 200 μm, from said surface.

41. The method according to any of the claims 39-40, wherein said volume to be treated or disinfected has a surface, and wherein said at least one focal volume is a distance of 1-500 μm, more preferred 5-300 μm, preferably 10-200 μm, more preferred 40-100 μm, from said surface.

42. The method according to any of the claims 39-41, wherein said electromagnetic radiation is generated by a laser.

43. The method according to claim 42, wherein said laser is operated in a continuous or pulsed mode.

44. The method according to any of the claims 39-43, wherein said at least one focal volume is moved within said volume to be treated or disinfected with a velocity allowing said electromagnetic to provide sufficient energy in said at least one focal volume to eradicate or harm said bacteria while providing insufficient energy to substantially harm said cells.

45. The method according to any of the preceding claims, wherein said volume to be treated or disinfected comprises at least part of a wound, such as a chronic wound.

46. The method according claim 45, wherein said the electromagnetic radiation is focused inside said wound.

47. The method according to any of the preceding claims, wherein said method comprises changing the position of said focal volume inside said volume to be treated or disinfected in a helical and/or zigzag pattern.

48. The method according to any of the preceding claims, wherein said method comprises changing the position of said focal volume, allowing said focal volume to travel in lines through said volume to be treated or disinfected with a determined spacing between said lines.

49. The method according to claim 48, wherein said determined spacing is 1-200 μm, preferably 2-100 μm, more preferred 3-50 μm, preferably 5-40 μm, more preferred 10-30 μm, preferably 15-25 μm, more preferred about 20 μm.

50. The method according to any of the preceding claims, wherein said electromagnetic radiation has a wavelength of 100-3000 nm, preferably 200-2500 nm, more preferred 300-2000 nm, preferably 500-1500 nm, more preferred 700-1400 nm, preferably 800-1300 nm, more preferred 900-1200 nm, preferably 1000-1125 nm, more preferred 1025-1100 nm, preferably 1050-1080 nm, more preferred 1060-1070 nm, preferably about 1064 nm.

51. The method according to any of the preceding claims, wherein said electromagnetic radiation is provided as electromagnetic pulses.

52. The method according to claim 51, wherein said electromagnetic pulses have duration of 0.1-20 ns, more preferred 0.5-10 ns, preferably 1-8 ns, more preferred 2-6 ns, preferably 3-5 ns, more preferred about 4 ns.

53. The method according to claim 51 or 52, wherein the electromagnetic pulses have duration sufficient to eradicate or harm bacteria while having duration insufficient to substantially harm cells.

54. The method according to any of the claims 51-53, wherein each of said electromagnetic pulses provides an amount of energy of 1-10.000 nJ, preferably 5-5.000 nJ, more preferred 10-2500 nJ, preferably 20-1000 nJ, more preferred 30-500 nJ, preferably 40-100 nJ, more preferred about 50 nJ in each of said at least one focal volume.

55. The method according to any of the claims 51-54, wherein each of said electromagnetic pulses provides an amount of energy of less than 10.000 nJ, preferably less than 5.000 nJ, more preferred less than 2500 nJ, preferably less than 1000 nJ, more preferred less than 500 nJ, preferably less than 100 nJ, more preferred about less than 50 nJ in each of said at least one focal volume.

56. The method according to any of the claims 51-55, wherein said electromagnetic pulses are provided with a frequency of 1-100 kHz, more preferred 5-50 kHz, preferably 10-40 kHz, more preferred 15-30 kHz, preferably about 20 kHz.

57. The method according to any of the claims 51-56, wherein the electromagnetic pulses are focused inside said volume to be treated or disinfected with a distance of 1-200 μm, preferably 2-100 μm, more preferred 3-50 μm, preferably 5-40 μm, more preferred 10-30 μm, preferably 15-25 μm, more preferred about 20 μm between said pulses.

58. The method according to any of the preceding claims, wherein said focal volume is moved around in said volume to be treated or disinfected, thereby providing treatment and/or disinfection of all or substantially all of said volume to be treated or disinfected.

59. The method according to any of the preceding claims, wherein said focal volume is moved around multiple times in said volume to be treated or disinfected in multiple passes.

60. The method according to any of the preceding claims, wherein the contents of said volume comprising bacteria in the vicinity of cells is substantially solid or non-liquid or non-fluid.
